(19) United States
(12) Patent Application Publication
(10) Pub. No.: US 2020/0068936 A1
(43) Pub. Date: Mar. 5, 2020

(54) LINEXUS COMPOSITION

(71) Applicant: DuraScience Inc., New York, NY (US)

(72) Inventor: James Nitit Mah, New York, NY (US)

(73) Assignee: DuraScience Inc., New York, NY (US)

(21) Appl. No.: 16/679,283

(22) Filed: Nov. 10, 2019

(51) Int. Cl. A23L 33/105; A61K 36/287; A61K 36/736; A61K 36/746

(52) U.S. Cl. CPC A23L 33/105; A61K 36/746; A61K 36/736; A61K 36/287

(57) ABSTRACT

In an embodiment, a nutritional supplement composition is provided. The nutritional supplement composition is provided a combination of 100 mg of chrysanthemum extract from the chrysanthemum indica strain, 40 mg of acerola cherry extract from the malpighia glabra strain and 40 mg noni extract from the morinda citrifolia strain. The nutritional supplement is in the form of a compound known as LINEXUS™.

### BACKGROUND

In the past, we commonly used all parts of natural foods when we consumed a meal. Human eating patterns naturally accessed nutrients available from what was eaten and very little nutrients were wasted as a result. For example, when eating fish, the muscular parts of the fish were consumed along with other parts such as connective tissue. When eating plants, the edible portion of the plant was consumed in its entirety. Our bodies would then filter out what was not consumable, such as the outer shell of a kernel of corn for example. As this occurred, our bodies would extract what nutrients could be found in the food to sustain and grow the human form. This allowed use of local foods to provide nutrients which were available, but was somewhat limited in that foods not available locally could not be used in most cases. Over time, we learned to travel and to ship food from where it was produced to where it could be consumed. Additionally, we learned to prepare foods, removing portions of the food which were either inedible, or undesirable. Removing the inedible parts typically provided some benefit, while parts that were simply undesirable sometimes held valuable ingredients.

Chrysanthemum is a flowering plant found throughout China which is often used for tea.

Acerola cherry is a flowering plant native to the Americas, particularly in the areas from the southern end of the United States through Central America to Brazil.

The morinda citrifolia strain is a fruit-bearing tree in the coffee family, Rubiaceae. Its native range extends across Southeast Asia and Australasia. It is often referred to as noni, and is considered to have an exceptionally unpleasant smell and taste.

### SUMMARY OF THE INVENTION

A nutritional supplement composition is provided including a chrysanthemum extract from the chrysanthemum indica strain, acerola cherry extract from the malpighia glabra strain and noni extract from the morinda citrifolia strain. The nutritional supplement is in the form of a compound known as LINEXUS™.

The foregoing, and other features and advantages of various embodiments of the invention, will be apparent from the following, more particular description of the embodiments of the invention, any accompanying drawings, and the claims.

### DETAILED DESCRIPTION

A composition is provided as LINEXUS™. The specific embodiments described in this document represent exemplary instances of the present invention, and are illustrative in nature rather than restrictive.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments.

Chrysanthemum indicum strain appears to exhibit a variety of useful and beneficial health effects. Chrysanthemum has been variously described as antiphlogistic, blood tonic, depurative, febrifuge and vulnerary, providing a variety of benefits. It has been used to treat eye ailments, for example. It has also been used in relation to gonorrhoea. Moreover, chysanthemum has been used for treatment of headaches and migraine headaches in particular. Also, an essential oil obtained from chysanthemum is active on the brain center affected by Parkinson's disease.

The flowers in particular have been found by various researchers to be aperient, bitter, hypotensive, stomachic and vasodilating. They exhibit an antibacterial action in some cases, inhibiting growth of Staphylococcus, E. coli, Streptococcus, C. diphtheriae, and Bacillus dysenteriae. The flowers are used in the treatment of issues as diverse as furuncle, scrofula, deep-rooted boils, inflammation of the throat, eyes and cervix, eczema, itchiness of the skin and hypertension. Some research reports a rejuvenating effect of the flowers over time as well. Other research has shown chrysanthemum used to treat effects such as chest pain (angina), high blood pressure, type 2 diabetes, dizziness and swelling. Thus, chrysanthemum has been shown to provide a wide array of potential health benefits.

Acerola cherry of the mappighia glare strain is known to have a variety of beneficial effects. Traditionally, the fruits have been used to treat dysentery, diarrhea, and liver disorders. Other ethnobotanical uses include as an astringent and for fever. The fruits also appear to have some antioxidant properties. The fruit is a relatively small red berry-sized fruit which often includes Vitamin C content on the order of sixty-five (65) times as much Vitamin C as is typically found in an orange.

The main micronutrients of Morinda citrifolia include vitamin C, niacin (vitamin B3), iron and potassium. Morinda citrifolia fruit contains a number of phytochemicals, including flavonoids, iridoids, fatty acids, lignans, scopoletin, catechin, oligo- and polysaccharides, beta-sitosterol, damnacanthal, and alkaloids.

Morinda citrifolia (Noni) has been extensively used in folk medicine by Polynesians for over 2,000 years. It has been reported to have broad therapeutic effects, including anticancer activity, in both clinical practice and laboratory animal models. The mechanism for these effects remains unknown.

Green fruit, leaves, and root or rhizomes of noni have been used in Polynesian cultures as a general tonic. It is reported that individuals take noni by mouth for colic, convulsions, cough, diabetes, painful urination, stimulating menstrual flow, fever, liver disease, constipation, vaginal discharge during pregnancy, malarial fever, and nausea. The leaves have been used in medicines for rheumatic aches and swelling of the joints, stomachache, dysentery, and swelling caused by a parasitic infection called filariasis. The bark has been used in a preparation to aid childbirth. The fruit juice is used for arthritis, diabetes, high blood pressure, muscle aches and pains, menstrual difficulties, headaches, heart disease, AIDS, cancers, gastric ulcers, sprains, depression, senility, poor digestion, atherosclerosis, circulation problems, and drug addiction. None is also used for smallpox, enlarged spleen, swelling, asthma, arthritis and other bone and joint problems, cancer, cataracts, colds, depression, digestive problems, and gastric ulcers. Other uses include high blood pressure, infections, kidney disorders, migraine headache, premenstrual syndrome, stroke, pain, and sedation.

The benefit of the composition includes restoring and enhancing cells. Our cells constantly deteriorate in daily life. If there is no support to restore the deterioration, cellular damage will affect the body functions. LINEXUS™ composition is a supplementary factor that can restore the cells to the body balance without drug and substance usage. When cells are restored, in the next process, LINEXUS™ composition may accordingly boost cell strength, equivalent to increasing the effectiveness and the number of cells, while maximizing physical and mental abilities After the cellular restoration and boosting the cell strength, the next challenge is the protection to maintain the long-lasting quality of the cells. LINEXUS™ dietary supplement may help to protect and delay the cellular deterioration. This will also result in improving the immune system, as the immune system no longer needs to activate against inflammation or other forms of deterioration. When the cell functions are systematically restored, boosted and protected, LINEXUS™ dietary supplement may directly enhance physical and mental capacity to go beyond limits at each age and to live life fully as the pace of life requires a strong response to maintain youth and vigor.

The health benefits of LINEXUS™ are also potentially of value to children, and help to develop the strength of new cells and protect cells from early deterioration. Both are beneficial to the development of body, intelligence, memory, and positive emotions. The physical results may include i) restore cells for normal growth in each stage, ii) recover from illness, iii) build muscle, v) boost the immune system, vi) reduce the risk of the incidence of disease, vii) promote growth to maximum effectiveness, viii) adjust height, ix) improve body growth, x) efficient immunity and xi) increase energy. The wound and injury healing properties are potentially of particular assistance to children, as the nutrients provided assist specifically with structures often injured in sports or other childhood activities.

The composition also helps to restore the old damaged cells, repair damaged body tissues, delay the cellular deterioration, inhibit free radicals, and help the process of skin cell renewal in adults for glowing skin and younger appearance. Providing building blocks as previously mentioned contribute to enhancing balance, reducing exhaustion from stress and demands of healing, improving concentration and work efficiency, reducing healing time and longer term pain, and boosting the immune system to stay healthy

Physical results may also include: a radiant and vibrant skin, younger appearance; reducing fatigue at work; restoring the reproductive system; refreshing the body; boosting the immune system; protecting against free radical formation; and preventing premature aging.

The compounds are typically dried and ground or milled to small particles, and subsequently combined in the desired ratio. The combination may remain as a powder to be added to food or pressed in a tablet. Alternatively, the compounds may be mixed in an aqueous or other liquid solution and thereby provided in liquid form.

In an embodiment, a preferred dosage, the total compound blended is 180 mg, pressed into a single tablet. The compound may be sold under the LINEXUS™ name. The dosage may include a combination of 100 mg of chrysanthemum extract from the chrysanthemum indica strain, 40 mg of acerola cherry extract from the malpighia glabra strain and 40 mg noni extract from the morinda citrifolia strain. However, other ratios of the ingredients may be provided, and smaller or larger dosages may be used for particular applications.

### Study of LINEXUS™ Effectiveness

A study of users of a trial formulation was conducted. The study focused initially on dermatological factors, but also considered other possible results. Results were broken down into dermatological factors and other potentially related factors. Participants reported the following results:

Eighty-six percent (86%) of participants reported better bone density, with an average reported improvement of approximately twenty percent (20%). This appeared to relate to disruption in bone-resorbing activity and improving phytoestrogenic effects.

Approximately eighty-eight percent (88%) of participants reported a substantial decrease in bone fracture, with a reported decrease typically of eighty-five percent (85%).

Eighty-three percent (83%) of participants reported significant decreases in gout attacks or flareups, typically reporting about a seventy-five (75%) decrease.

Also, approximately eighty-six percent (86%) of participants reported decreases in size of gouty tophi after long term use related to urate crystal reabsorption, with an observed decrease in size of approximately eighty percent (80%).

Blood pressure reduction of approximately ten percent (10%) was also observed in ninety percent (90%) of participants after long term use.

Approximately ninety percent (90%) of study participants observed decreased blood uric level and corresponding uric acid secretion, with a typical measurement of about thirty percent (30%) decrease. This appears to relate to inhibition of the xanthine oxidase enzyme.

Ninety percent (90%) of participants reported decreased pain from osteoarthritis, with a typical observation of eighty percent (80%) less pain.

Similarly, approximately eighty-one percent (81%) of participants reported decreases in severity and duration of joint pain, suggesting anti-inflammatory effects.

One skilled in the art will appreciate that although specific examples and embodiments of the system and methods have been described for purposes of illustration, various modifications can be made without deviating from present invention. For example, embodiments of the present invention may be applied to many different types of databases, systems and application programs. Moreover, features of one embodiment may be incorporated into other embodiments, even where those features are not described together in a single embodiment within the present document.

What is claimed is:

1. A nutritional composition consisting of:
chrysanthemum extract from the chrysanthemum indica strain;
acerola cherry extract from the malpighia glabra strain; and
noni extract from the morinda citrifolia strain.

2. The composition of claim 1, wherein:
the chrysanthemum extract is in the amount of 100 mg,
the acerola cherry extract is in the amount of 40 mg, and
the noni extract is in the amount of 40 mg.
